(19) United States
(12) Patent Application Publication
(10) Pub. No.: US 2025/0111930 A1
(43) Pub. Date: Apr. 3, 2025

(54) SYSTEMS AND METHODS FOR MEDICAL IMAGE ANNOTATION AND ANATOMICAL LANDMARK HEIGHT POSITIONING

(71) Applicant: GE Precision Healthcare LLC, Waukesha, WI (US)

(72) Inventors: Olivier Gérard, Oslo (NO); Ella Sokulin, Kiryat Tivon (IL); Kjetil Viggen, Trondheim (NO); Vipin Koothur, Oslo (NO)

(21) Appl. No.: 18/477,440

(22) Filed: Sep. 28, 2023

(51) Int. Cl. G16H 30/40; G06T 7/00; G06V 20/70; G16H 30/20

(52) U.S. Cl. CPC G16H 30/40; G06T 7/0012; G06V 20/70; G16H 30/20; G06T 2207/10132; G06T 2207/20101; G06T 2207/30048; G06V 2201/07

(57) ABSTRACT

Methods and systems are herein provided for image annotation for anatomical landmark measurement. In one example, a method comprises obtaining an image dataset acquired by an imaging system; displaying an image of the image dataset within a graphical user interface (GUI) on a display device communicatively coupled to the imaging system; determining first and second anatomical landmark points of the image; generating an annotation overlay based on at least in part on the first and second anatomical landmark points, wherein the annotation overlay comprises a first line connecting the first and second anatomical landmark points, a second line perpendicular to the first line, and a third line perpendicular to the second line, wherein the second line connects the first line to the third line and defines a height of the third line above the first line; and displaying and saving the annotation overlay.

### FIELD

The present description relates generally to methods and systems for medical image annotation, and more particularly to determining a height and position of an anatomical landmark.

### BACKGROUND

Medical ultrasound is an imaging modality that employs ultrasound waves propagating through the internal structures of a body of a patient to produce a corresponding image. For example, an ultrasound probe comprising a plurality of transducer elements emits ultrasonic waves which reflect or echo, refract, or are absorbed by structures in the body. Medical ultrasound modalities, such as echocardiograms, are also used prior to, during, and/or after procedures to image internal structures relevant to the procedure. For example, for certain cardiac procedures such as minimally invasive cardiac surgeries that employ use of a catheter and/or other devices, ultrasound images such as echocardiogram images may be used to guide positioning of the catheter.

### BRIEF DESCRIPTION

In one embodiment, a method comprises obtaining an image dataset acquired by an imaging system; displaying an image of the image dataset within a graphical user interface (GUI) on a display device communicatively coupled to the imaging system; determining first and second anatomical landmark points of the image; generating an annotation overlay based on at least in part on the first and second anatomical landmark points; and displaying and saving the annotation overlay.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 schematically shows an imaging system according to an embodiment.

FIG. 2 shows a block diagram of a processor of the imaging system of FIG. 1.

FIG. 3 shows a first example graphical user interface (GUI) displaying an image acquired by the imaging system of FIG. 1.

FIG. 4 shows a second example GUI in which a first mitral valve annulus point is indicated.

FIG. 5 shows the second example GUI displaying an annotation overlay with a first height displayed.

FIG. 6 shows the second GUI displaying the annotation overlay with a second height displayed.

FIG. 7 shows a third example GUI displaying a saved annotation overlay.

FIG. 8 shows a three-dimensional ultrasound image displayed in a fourth GUI with an annotation overlay.

FIG. 9 shows a flowchart illustrating a method for generating an annotation overlay via user inputs.

FIG. 10 shows a flowchart illustrating a method for generating an annotation overlay via automated algorithms.

### DETAILED DESCRIPTION

The following description relates to various embodiments for systems and methods for annotation of medical images. More specifically, systems and methods are herein provided for medical image annotation for determining a transseptal puncture (TSP) position. Pre-operative, intra-operative, and post-operative imaging of relevant internal structures of a patient are often used to aid surgeons and other medical personnel in determining proper positioning of instruments, size of hardware, and the like. In minimally invasive cardiac procedures, a device is maneuvered through the venous system to the heart. In some examples, such as procedures like mitral valve replacements, patent foramen ovale closures, left atrial appendage occlusions, and more, the device is maneuvered into the left atrium and then crosses the atrial septum to the right atrium via a TSP. TSPs demand precise positioning in order for the device to properly continue to be maneuvered through the vasculature to perform the respective procedure.

Pre-operative imaging is recommended for patients undergoing TSP as a portion of a procedure in order to determine the interatrial septal anatomy, including position of a portion of the interatrial septum that is usable for TSP, as well as for detection of septal abnormalities. Pre-operative imaging may include computerized tomography (CT) images, ultrasound images such as a transesophageal echocardiogram (TEE), fluoroscopic images, and the like. Intra-operative imaging is also recommended to allow for visualization of the interatrial septum, especially to determine positioning of the device, such as a sheath, in real time (e.g., without intentional delay). As fluoroscopic images do not show interatrial septal anatomy well and intra-operative CT is impractical for intraoperative purposes, echocardiograms, TEE or intracardiac echocardiogram (ICE), have been used to visualize the relevant anatomy as well as the position of the device. For example, the position of the device in real-time may be determined by visualization of a “tent” in the interatrial septum, the tent being a protrusion of the septum from pressure from the device on the septum.

The position of the TSP is typically planned to a height above the patient's mitral valve annulus, called the height of the puncture. A common puncture height is 4 cm above the mitral valve annulus, though individual patient anatomy may result in a different puncture height being recommended. For both pre-operative and intra-operative imaging, measuring the ideal puncture height is performed through interactive annotation of medical images, such as echocardiogram images. The interactive annotation is often time consuming and potentially inaccurate if the annotated measurement line is improperly positioned or angled. Annotation may comprise drawing multiple lines onto an image in a graphical user interface (GUI), and if any of the lines are repositioned, the other lines may also demand repositioning, which is time consuming for the user. Further, in order to properly define the puncture height, the level of the puncture (e.g., in some examples measured at a tenting position) may be drawn parallel to the mitral valve annulus and therefore an annotated line therebetween indicating the puncture height, may be perpendicular to the mitral valve annulus. Inaccurate positionings of lines and therefore inaccurate measurements may lead to improper placement of the device within the atria.

Systems and methods for annotation of medical images to determine position of a TSP, or other application, are herein presented that addresses the aforementioned issues. An annotation tool within a GUI may allow a user to indicate a first anatomical landmark in a medical image with user inputs. Based on the user inputs defining the first anatomical landmark and a height (e.g., a puncture height), which may be predefined via user input(s), and/or determined via detection of a landmark such as a tenting position, an annotation overlay may be displayed. The annotation overlay may indicate a position, such as a position of a puncture site, and the height may be editable via user inputs without altering the annotated position of the first anatomical landmark. The annotation tool may be used for intraoperative images as well as preoperative and/or postoperative images. In a use-case scenario, live intraoperative images may be annotated to determine a current position of a device based on a visualized tenting. Portions of the methods herein described, including determining positions of the anatomical landmark, such as a mitral valve annulus, and determining the height based on another anatomical landmark, such as a tenting position, may be automated. In this way, the time spent by the user may be reduced. Additionally, as the annotation overlay defines a perpendicular set of lines, accuracy may be increased.

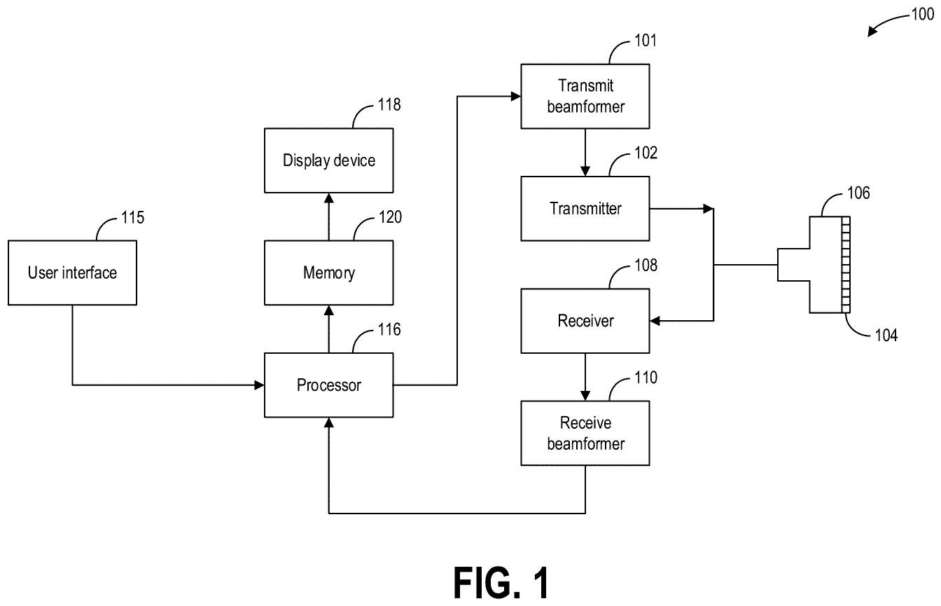

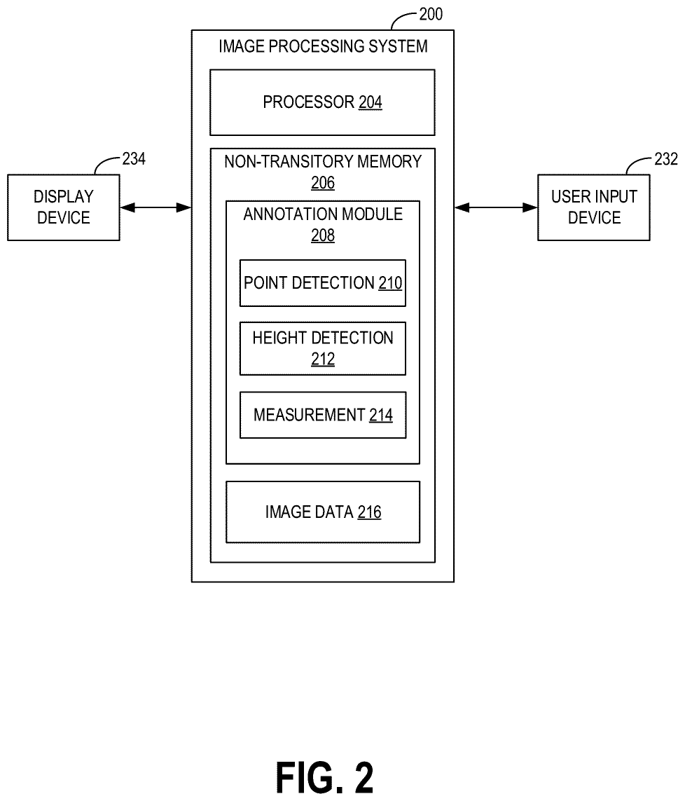

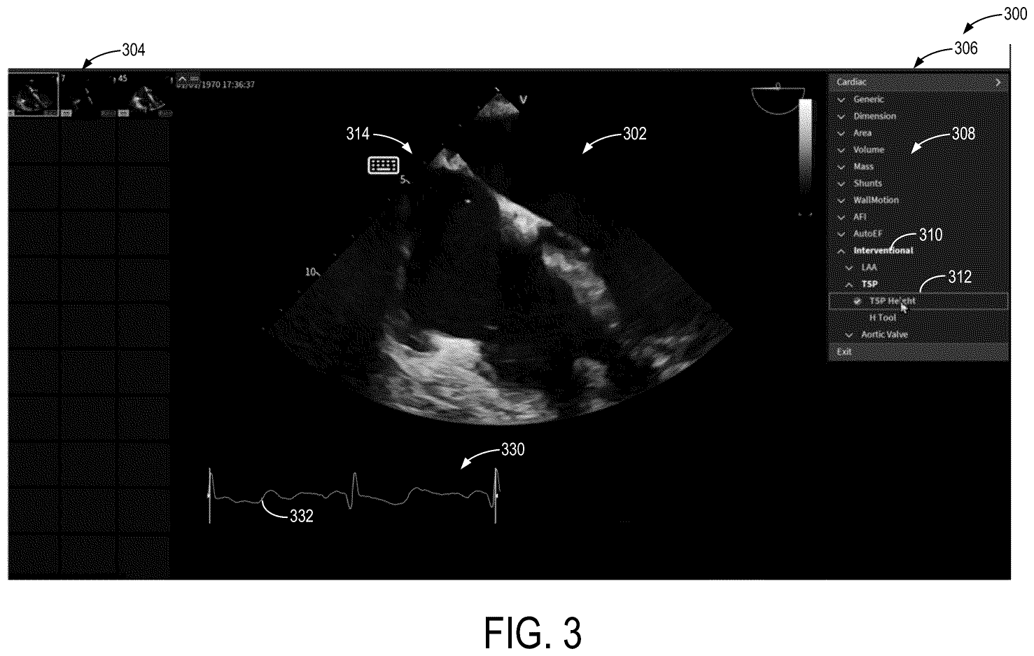

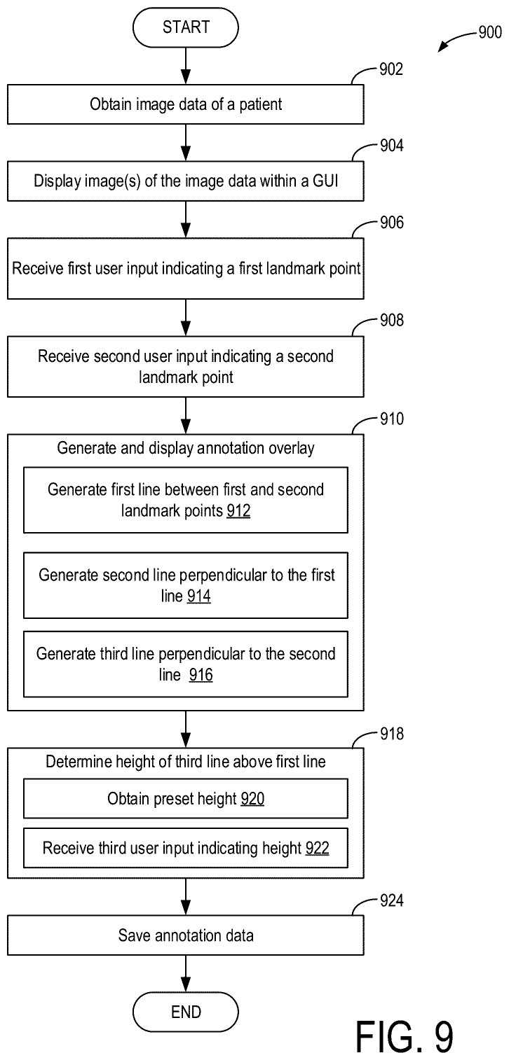

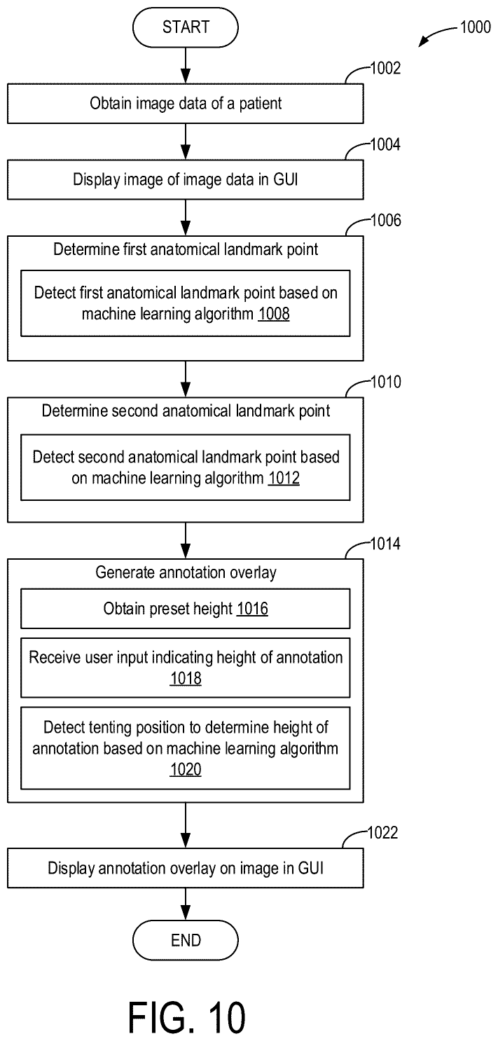

An example imaging system including a display device and an imaging processing system are shown in FIG. 1. The imaging processing system, as shown in FIG. 2, may be used to analyze and annotate images acquired by the imaging system of FIG. 1. FIGS. 3-8 show example GUIs with various annotations and overlays displayed on medical images that may be acquired by the imaging system of FIG. 1. FIG. 9 illustrates a method for generating a TSP annotation overlay via user inputs and FIG. 10 illustrates a method for automated determination of a TSP annotation overlay.

The methods herein described may be implemented on various types of medical images, for example two-dimensional (2D) and/or three-dimensional (3D) ultrasound images like echocardiograms, CT images, and more. Such images may be acquired by an imaging system, such as an ultrasound system or CT system. An example system for implementation of the methods is shown in FIG. 1. Therein, a block diagram of a system 100 is depicted, according to one embodiment. In the illustrated embodiment, the system 100 is an imaging system and, more specifically, an ultrasound imaging system 100. As shown, the ultrasound imaging system 100 includes multiple components. The components may be coupled to one another to form a single structure. In one example, the ultrasound imaging system 100 is a unitary system that is capable of being moved (e.g., portably) from room to room. For example, the ultrasound imaging system 100 may include one or more components configured to couple the ultrasound imaging system 100 to a wheeled cart. However, in other examples, at least portions of the ultrasound imaging system 100 may be configured to remain stationary and/or fixed in place.

After the elements 104 of the probe 106 emit pulsed ultrasonic signals into a body (of a patient), the pulsed ultrasonic signals are back-scattered from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. Additionally, transducer element 104 may produce one or more ultrasonic pulses to form one or more transmit beams in accordance with the received echoes.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. In one embodiment, data acquired via ultrasound system 100 may be used to train a machine learning model. A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment, the demodulation can be carried out earlier in the processing chain. The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/see while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data, for example by augmenting the data as described further herein, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

In various embodiments of the present disclosure, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

After performing a two-dimensional ultrasound scan, a block of data comprising scan lines and their samples is generated. After back-end filters are applied, a process known as scan conversion is performed to transform the two-dimensional data block into a displayable bitmap image with additional scan information such as depths, angles of each scan line, and so on. During scan conversion, an interpolation technique is applied to fill missing holes (e.g., pixels) in the resulting image. These missing pixels occur because each element of the two-dimensional block should typically cover many pixels in the resulting image. For example, in current ultrasound imaging systems, a bicubic interpolation is applied which leverages neighboring elements of the two-dimensional block.

Ultrasound images acquired by ultrasound imaging system 100 may be further processed. In some examples, ultrasound images produced by ultrasound imaging system 100 may be transmitted to an image processing system, where in some examples, the ultrasound images may be analyzed to determine anatomical landmark positions (e.g., mitral valve annulus points). In some examples, machine learning algorithms may be employed for analysis of ultrasound images. As an example, diverse views from various ultrasound images showing mitral valve annulus points and tenting positions annotated and trained to a segmentation based network. Based on the segmentation based network, the acquired ultrasound images may be annotated in an automated manner, whereby mitral valve annulus points and tenting positions are detected based on the trained segmentation based network.

Although described herein as separate systems, it will be appreciated that in some examples, ultrasound imaging system 100 includes an image processing system, for example as part of the processor 116. In other examples, ultrasound imaging system 100 and the image processing system may comprise separate devices. For example, the image processing system may be disposed at a workstation.

It should be understood that while an ultrasound imaging system is described herein, this is a non-limiting example of an imaging system as may be used to acquire medical images upon which the methods herein may be implemented. Other imaging systems, such as CT systems, magnetic resonance imaging (MRI) systems, and the like may be used without departing from the scope of this disclosure.

Referring to FIG. 2, image processing system 200 is shown, in accordance with an exemplary embodiment. In some examples, image processing system 200 is incorporated into the ultrasound imaging system 100. For example, the image processing system 200 may be provided in the ultrasound imaging system 100 as the processor 116 and memory 120. In some examples, at least a portion of image processing system 200 is disposed at a device (e.g., edge device, server, etc.) communicably coupled to the ultrasound imaging system 100 via wired and/or wireless connections. In some examples, at least a portion of image processing system 200 is disposed at a separate device (e.g., a workstation) which can receive images/maps from the ultrasound imaging system or from a storage device which stores the images/data generated by the ultrasound imaging system. Image processing system 200 may be operably/communicably coupled to a user input device 232 and a display device 234. The user input device 232 may comprise the user interface 115 of the ultrasound imaging system 100, while the display device may comprise the display device 118 of the ultrasound imaging system 100, at least in some examples.

Image processing system 200 includes a processor 204 configured to execute machine readable instructions stored in non-transitory memory 206. Processor 204 may be single core or multi-core, and the programs executed thereon may be configured for parallel or distributed processing. In some examples, the processor 204 may optionally include individual components that are distributed throughout two or more devices, which may be remotely located and/or configured for coordinate processing. In some examples, one or more aspects of the processor 204 may be virtualized and executed by remotely-accessible networked computing devices configured in a cloud computing configuration.

Non-transitory memory 206 may store an annotation module 208. The annotation module 208 may generate an annotation overlay within a graphical interface. The annotation module 208 may store a plurality of models. In some examples, generation of the annotation overlay may be automated based at least in part on algorithms of point detection model 210 and height detection model 212. The point detection model 210 may detect one or more first anatomical landmark positions and the height detection model 212 may detect a second type of anatomical landmark position to inform the annotation module 208 of anatomical landmarks in order to generate the annotation overlay based on those landmarks. In some examples, the annotation module 208 may further include measurement model 214 which may determine a height measurement. In some examples, a height may be indicated via user inputs and/or may be determined automatically via the height detection model 212. An exact or near exact height of the second anatomical landmark position may be determined by the measurement model 214. The height of the second anatomical landmark may be measured in relation to the one or more first anatomical landmark positions. A use case example, as is herein presented, includes mitral valve annulus points as the first anatomical landmarks and a TSP puncture position as the second anatomical landmark, the height being a puncture height above the mitral valve annulus.

Non-transitory memory 206 may further store image data 216, such as ultrasound images captured by the ultrasound imaging system 100 of FIG. 1. The images of the image data 216 may comprise images that have been acquired by the ultrasound imaging system 100 with different anatomical features (e.g., different views of the heart) and/or different image quality. Further, image data 216 may store images, ground truth output, iterations of machine learning model output, and other types of image data that may be used to train the annotation module 208.

In some embodiments, the non-transitory memory 206 may include components disposed at two or more devices, which may be remotely located and/or configured for coordinated processing. In some examples, one or more aspects of the non-transitory memory 206 may include remotely-accessible networked storage devices configured in a cloud computing configuration.

User input device 232 may comprise one or more of a touchscreen, a keyboard, a mouse, a trackpad, a motion sensing camera, or other device configured to enable a user to interact with and manipulate data within image processing system 200. In some examples, user input device 232 may enable a user to make annotation inputs and otherwise interact with GUIs displayed on the display device 234. For example, the user input device 232 may enable a user to make an annotation inputs indicating a first mitral valve annulus point and a second mitral valve annulus point which may allow for generation of an annotation overlay. Further, the user input device 232 may enable a user to edit a height of the annotation overlay, for example via drag and release inputs.

Display device 234 may include one or more display devices utilizing virtually any type of technology. In some examples, display device 234 may comprise a computer monitor, and may display images such as ultrasound images acquired with ultrasound imaging system 100 within a GUI. Display device 234 may be combined with processor 204, non-transitory memory 206, and/or user input device 232 in a shared enclosure, or may be peripheral display devices and may comprise a monitor, touchscreen, projector, or other display device known in the art, which may enable a user to view images produced by an imaging system such as ultrasound imaging system 100, and/or interact with various data stored in non-transitory memory 206. In some examples, the display device 234 may be configured to display images stored in memory by a storage database, such as a picture archive and communication system (PACS). In other examples, the display device 234 may be configured to display images acquired in real-time, for example when the display device 234 is combined in the shared enclosure with the imaging system, the display device 234 may display images acquired in real-time. In some examples, the display device 234 may be configured to display both image stored in a database and images in real-time. In this way, both pre-operative images stored in PACS and intra-operative images acquired during a procedure may be annotated on the same or different display devices.

It should be understood that image processing system 200 shown in FIG. 2 is for illustration, not for limitation. Another appropriate image processing system may include more, fewer, or different components.

FIG. 3 shows an example first GUI 300. The first GUI 300 may be displayed on a display device, such as display device 118 of FIG. 1 and/or display device 234 of FIG. 2. Images displayed within the first GUI 300 may be acquired by an imaging system communicably coupled to the display device on which the first GUI 300 is displayed, such as the ultrasound imaging system 100 of FIG. 1. The first GUI 300 may display a first image 302, in some examples within a viewport 314 of the first GUI 300. A size and shape of the viewport 314 may be dependent upon type of image. For example, the first image 302 as displayed in the first GUI 300 may be an ultrasound echocardiography image and therefore the viewport 314 may be shaped as a triangular, pie-shape, corresponding to distribution of ultrasound waves from the transducer. Other types of images may be displayed in differently shaped viewports.

The first GUI 300 may include an image menu 304 through which a user may select one of a plurality of images to be displayed within the viewport 314. The plurality of images may include images from different scans of a patient, different views (e.g., from different axes) of the same scan, and the like. As an example, for a TEE scan, the plurality of images available may include a mid-esophageal four chamber, a mid-esophageal 2 chamber, a mid-esophageal long axis, a transgastric basal short axis, and a transgastric mid-papillary short axis. As another example, the plurality of images available may include a TEE scan (e.g., all images of the TEE scan included therein), an ICE scan (e.g., all images of the ICE scan included therein), and a cardiac CT scan. As noted, the shape of the viewport 314 may depend on which of the plurality of images is selected.

The first GUI 300 may further include a tool menu 306. The tool menu 306 may include a plurality of scan types that are selectable, for example, in the first GUI 300 the scan type is selected as cardiac. The scan type may be dependent upon the selected image that is displayed, in some examples. The tool menu 306 may further include a plurality of tool types 308. The plurality of tool types 308 may include various annotation tools, measurement tools, parameter tools, and more. Each of the tool types 308 may be selectable to display a respective drop down menu. For example, the plurality of tool types 308 may include an automatic ejection fraction calculator tool menu that when selected displays one or more ejection fraction calculator tools. One of the plurality of tool types 308 may be an interventional tool type 310. The interventional tool type 310 may include various tool sub-types in a drop down menu that are relevant to one or more procedures, interventions, surgeries, etc. The various tool sub-types may include, as non-limiting examples, left atrial appendage (LAA), TSP, and aortic valve. Each of the sub-types, when selected via user input, may launch a respective drop down menu that displays respective tools thereof.

A first GUI tool of the interventional tool type 310 may include an annotation height tool 312. The annotation height tool 312, as will be further described below, may allow for determination and measurement of a height of an anatomical position (e.g., a TSP position) based on various anatomical landmarks determined either via user input or automated algorithms.

The first GUI 300 may also show a heart rhythm 330 in instances in which heart rhythm is catalogued for the selected and displayed image. The heart rhythm 330 as displayed may correspond to the displayed image. In some examples, a tracker 332 may indicate where in the heart rhythm the displayed image is. For example, heart rhythm may be monitored during echocardiogram ultrasound images and therefore when an echocardiogram image is selected to be displayed within the first GUI 300, the heart rhythm 330 may be displayed. As the displayed echocardiogram image moves through its captured timeframe, the tracker 332 may move along the heart rhythm 330. For cardiac CTs, however, where static images are acquired as opposed to echocardiograms which include motive images, heart rhythm may not be monitored and therefore the heart rhythm 330 may not be displayed.

While not explicitly shown in FIG. 3, the first GUI 300 may also include various demographics of a patient of which the first image 302 was acquired, including name, birthdate, age, and the like, as well as data of the first image, including scan type, parameters, date acquired, and more.

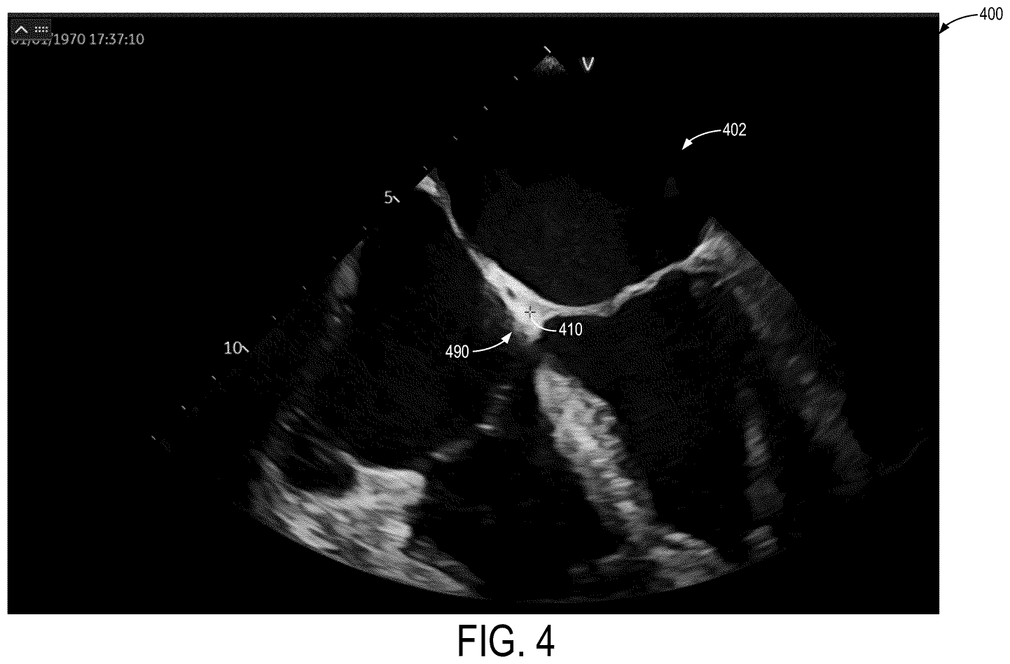

Turning to FIG. 4, a portion of an example second GUI 400 is shown. In some examples, the second GUI 400 may be the first GUI 300 described with respect to FIG. 3. The second GUI 400 may be displayed on a display device, such as the display device 118 of FIG. 1 and/or the display device 234 of FIG. 2 that is communicably coupled to an imaging system, such as the ultrasound imaging system 100 of FIG. 1, used to acquire images that are displayed within the second GUI 400.

Similar to as described with respect to FIG. 3, the second GUI 400 may display a second image 402. In some examples, the second image 402 may be acquired of a patient pre-operatively, intra-operatively, or post-operatively for a corresponding procedure. For example, the second image 402 may be acquired intra-operatively during a minimally invasive cardiac procedure and may be displayed by the display device in real-time and/or at a later time. In such examples, as is herein described, the second image 402 may comprise a plurality of anatomical features of the patient's heart. The plurality of anatomical features includes a mitral valve annulus, an interatrial septum, and the like. Each of the anatomical features may have one or more landmarks that are visible within the second image 402. The one or more landmarks may be annotated and/or analyzed in order to generate an annotation overlay and/or to determine a height.

The second image 402 may be annotated via various tools accessible through the second GUI 400 and/or via automated algorithms. As an example, a TSP annotation tool, also called a TSP height tool herein, may be accessible via the second GUI 400. When selected, the TSP height tool may allow the user to annotate the second image 402 to determine a proposed puncture height for a TSP.

Annotations to the second image 402 may include a first anatomical landmark point 410. The first anatomical landmark point 410 may be selected via a first type of user input via an annotation tool. In examples in which the annotation tool is a TSP annotation tool, the first anatomical landmark point 410 may indicate a position of a first side 490 of the mitral valve annulus. The first type of user input may be a left mouse click, a touchpad touch, a touch to a touchscreen, or the like. The first anatomical landmark point 410 may be displayed on the second image 402 as a cross hatch mark or other suitable point indicator. The first anatomical landmark point 410, in some examples, may be selectable to edit the position of the first anatomical landmark point 410 or to indicate a connected, second anatomical landmark point, as will be further described below.

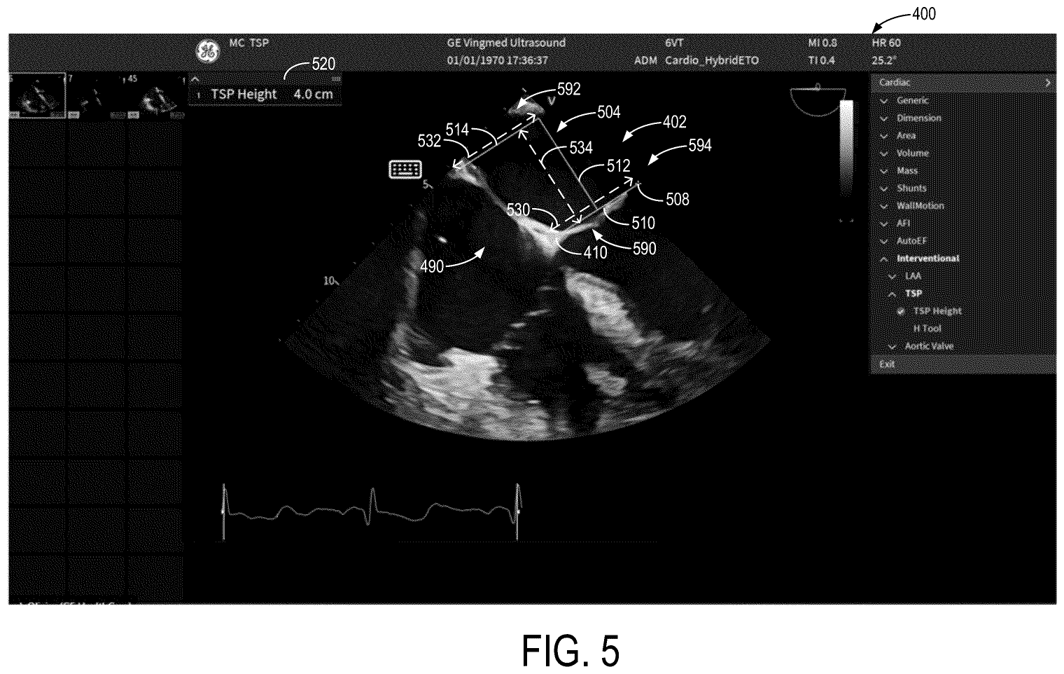

Referring now to FIG. 5, the second GUI 400 of FIG. 4 is shown including an annotation overlay 504. As noted above, the second GUI 400 may be displayed on a display device communicably coupled to an imaging system and images may be displayed within the GUI that, in some examples, are acquired by the imaging system. The second GUI 400 includes the annotation tool, accessible via a menu, as described with respect to FIG. 3. When selected, one or more landmarks may be indicated via user inputs. The first anatomical landmark point 410, as described in FIG. 4, may be indicated via the first type of user input. The annotation overlay 504 may comprise a plurality of lines, including a first line 510, a second line 512, and a third line 514.

A second anatomical landmark point 508 may be indicated via a second type of user input. In examples where the annotation tool is a TSP annotation tool, the second anatomical landmark point 508 may indicate a position of a second side 594 of the mitral valve annulus. In some examples, the second type of user input may be the same as the first type of user input. In other examples, the second type of user input may be different from the first type of user input, for example the second type of user input may be a cursor drag and mouse click/touchpad touch/finger touch release.

The first line 510 may be generated between the first anatomical landmark point 410 and the second anatomical landmark point 508. The first line 510 may be a straight line connecting the two landmark points, thereby indicating an estimated angle of the mitral valve annulus within the second image 402. The first line 510 may have a first length 530. At a midpoint of the first length 530, the second line 512 may be generated perpendicular to the first line 510. A first end 590 of the second line 512 may contact the midpoint of the first line 510. The third line 514 may be generated in contact with a second end 592 of the second line 512. The third line 514 may be perpendicular to the second line 512 and parallel to the first line 510. In some examples, the third line 514 may extend towards the first side 490 at least the same length as the first length 530. In other examples, the third line 514 may extend towards both the first side 490 and towards the second side 594. A second length 532 of the third line 514, in examples in which the third line 514 extends towards the first side 490 at least the same length as the first length 530, may be equal to the first length 530.

A third length 534 of the second line 512 that extends between the first line 510 and the third line 514 may be the height. This height may be the puncture height when the annotation tool is the TSP annotation tool. The height may be defined by a preset height saved in memory, for example 4 cm, via user inputs to a height element 520, and/or via user inputs changing the position of the third line 514. As an example, the third length 534 may be 4 cm (or proportional to 4 cm depending on the display parameters) initially based on a preset height. A user may input a different height into the height element 520 via user inputs, such as a combination of mouse clicks and keyboard touches, to define a second height of 3.5 cm. The third length 534 may change in real-time to 3.5 cm (or proportional to 3.5 cm) matching the defined second height. Changes to the height may not affect the position of the first line 510. As another example, the third length 534 may be changed via user inputs, such as a mouse click and drag of the third line 514. The height displayed in the height element 520 may update in real time as the third length 534 is changed. In this way, the height may be set to a desired height based on the patient's anatomy shown in the second image 402 and based on a height above the first anatomical landmark, as indicated by the third length 534. Further, the annotation overlay may be interactive based on user inputs and may update in real time in response to various user inputs.

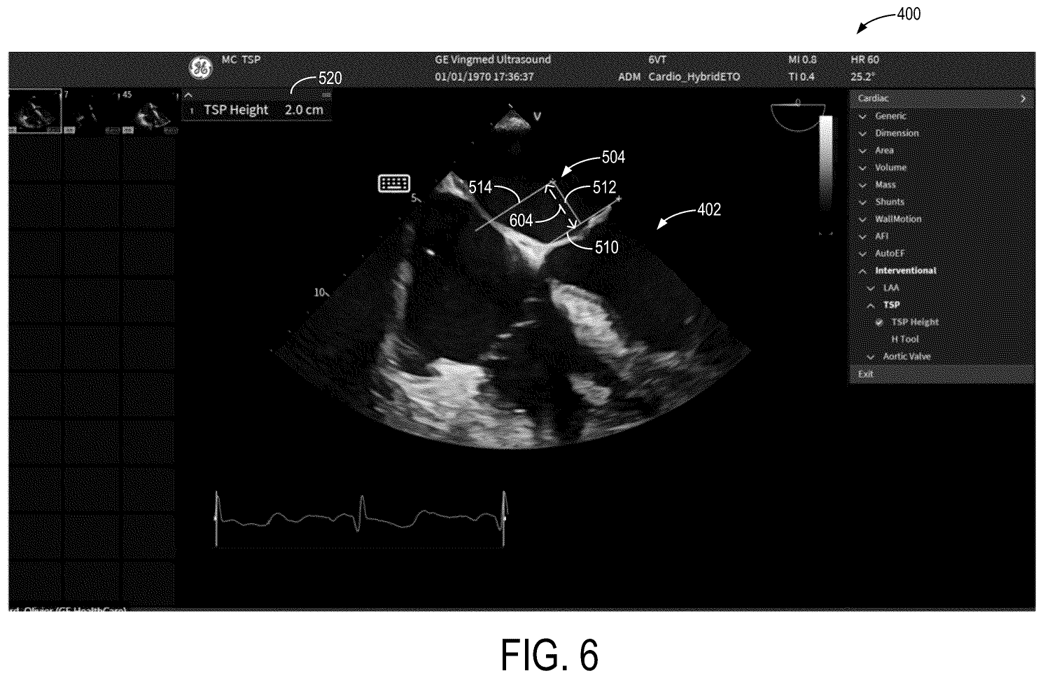

Turning briefly to FIG. 6, the annotation overlay 504 as displayed within the second GUI 400 with an altered third length 604 is shown. The altered third length 604 may extend between the first line 510 and the third line 514, just as the third length 534. As described, the third length 534 may become the altered third length 604 in response to one or more various user inputs. For example, the position of the third line 514 may be altered via a user input and/or the height within the height element 520 may be altered via a user input. The height displayed within the height element 520 in FIG. 5 is 4 cm and the third length 534 corresponds to the height of 4 cm. The height displayed within the height element 520 in FIG. 6 is 2 cm and the altered third length 604 may correspond to the height of 2 cm.

Returning to FIG. 5, in some examples, the image displayed within the second GUI 400 may be obtained intra-operatively. In the use case example presented of the TSP annotation tool, the tool may be used on images acquired during a minimally invasive cardiac procedure such that a tenting of the interatrial septum caused by the device that is to puncture through the septum is visualized. The height of the third line 514 may be aligned with the position of the tenting to define a puncture height (e.g., the third length 534) based on the position of the tenting. The height of the third line 514 may be aligned with the position of the tenting in an automated manner based on detection of the position of the tenting and/or based on user inputs changing the position of the third line 514.

The annotation overlay 504, when editable, may be displayed as a first color, indicating that the positions of the mitral valve annulus points and the third line are editable by the user. In some examples, the positions of the mitral valve annulus points and the height of the third line may be determined by one or more algorithms. The annotation overlay 504 may be generated based on the determined points and displayed with the first color. The various points and positions determined by the algorithm may be altered via user inputs to the GUI. The annotation overlay 504 may be saved to memory once the user determines that the positions and points are as desired.

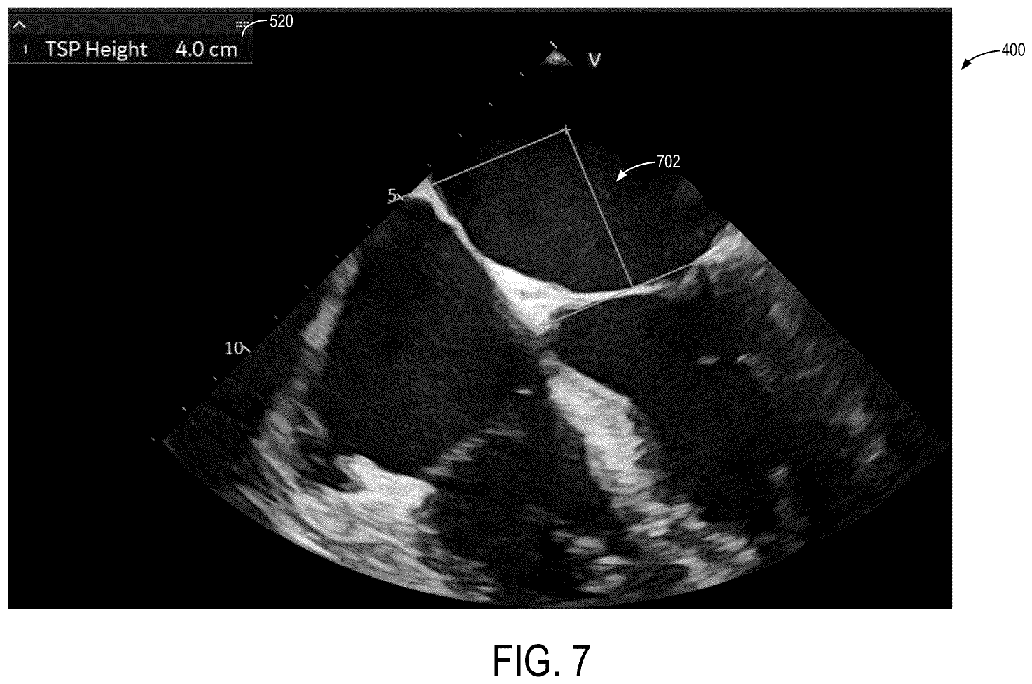

FIG. 7 shows the second GUI 400 with a saved annotation overlay 702. The saved annotation overlay 702 may be displayed within the second GUI 400 with a second color, different from the first color. The different colors may indicate to the user whether the annotation overlay is editable or whether it has been saved. For example, intra-operatively, the user may look at an annotated pre-operative image and compare it to an intra-operative image during an annotation. The annotation in the annotated pre-operative image may be saved and therefore displayed as the second color, indicating to the user that the puncture height of the annotation overlay is not editable. The different colors in this way may reduce the possibility of the user confusing the two images and therefore more accurately measuring a predicted and an actual puncture height.

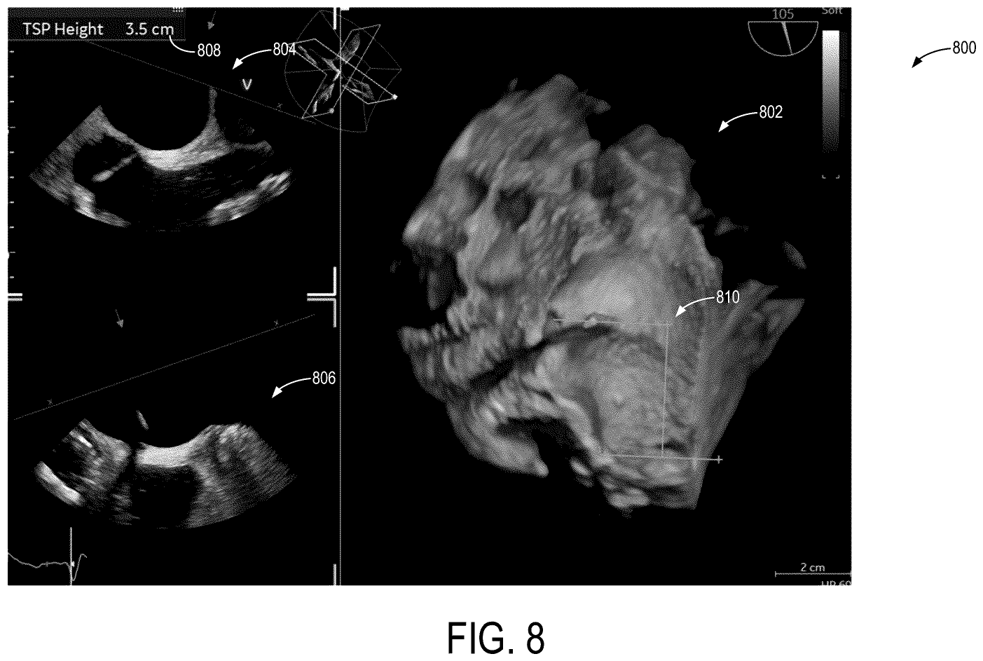

The first and second images 302, 402 herein shown are 2D images, though other types of images, including 3D images may also be displayed and annotated with the TSP height tool, and other tools. FIG. 8 shows an example of a 3D image 802 displayed within a third GUI 800. The 3D image 802 may be generated from one or more 2D images, such as first 2D image 804 and second 2D image 806. An annotation overlay 810 may be displayed over the 3D image 802 based on a plurality of points and/or positions indicating a position of a mitral valve annulus and a puncture height, similar to as described with respect to FIGS. 4-7. The height of the puncture height above the mitral valve annulus may be indicated by a height element 808, similar to as in the second GUI 400.

Turning now to FIG. 9, a flowchart illustrating a method 900 for generating an annotation overlay is shown. The method 900 will be described with relation to the systems depicted in FIGS. 1-2, but it should be understood that similar methods may be used with other systems without departing from the scope of this disclosure. The method 900 may be carried out via instructions stored in non-transitory memory of one or more computing devices. For example, method 900 may be carried out by the processor 204 based on instructions stored in non-transitory memory 206 of the image processing system 200 of FIG. 2.

At 902, method 900 includes obtaining image data of a patient. The image data may be 2D or 3D image data acquired by an imaging system. For example, the image data may be acquired by the ultrasound imaging system 100 of FIG. 1 and may comprise a plurality of 2D ultrasound images. In some examples, the plurality of 2D ultrasound images may be acquired over a period of time, for example images of a heart over a period of several heartbeats. In some examples, the image data may be obtained from a database that stores the data in memory, such as a PACS system. In other examples, the image data may be obtained by the imaging system in real-time and may therefore comprise live images.

At 904, method 900 includes displaying image(s) of the image data within a GUI, such as the first GUI 300 and/or the second GUI 400 described above. The GUI may be displayed by a display device in communication with the one or more computing devices, such as the display device 118 of FIG. 1 and/or the display device 234 of FIG. 2. The image(s) may be displayed within a viewport of the GUI, as described above.

At 906, method 900 includes receiving a first user input indicating a first landmark point. In some examples, the first landmark point may be a first mitral valve annulus point, wherein the first mitral valve annulus point indicates a position of a first side of the mitral valve annulus. The first side of the mitral valve annulus may serve as a first anatomical landmark for an annotation overlay. The first user input, such as a mouse click, may be inputted by a user input device, such as a mouse. The first user input may indicate a position of the first landmark point within a corresponding image as displayed within the GUI.

At 908, method 900 includes receiving a second user input indicating a second landmark point. In some examples, the second landmark point may be a second mitral valve annulus point, wherein the second mitral valve annulus point indicates a position of a second side of the mitral valve annulus. The second side of the mitral valve annulus may serve as a second anatomical landmark for the annotation overlay. The second user input may indicate a position of the second landmark point within the corresponding image as displayed within the GUI.

At 910, method 900 includes generating and displaying the annotation overlay based on the first and second user inputs. Generating the annotation overlay may comprise generating a first line between the first and second landmark points, as noted at 912. The first line may be a straight line connecting the first and second landmark points. In examples in which the first and second landmark points are mitral valve annulus points, the first line may approximate a position of the mitral valve annulus. Generating the annotation overlay may further comprise generating a second line perpendicular to the first line, as noted at 914. As is described with respect to FIG. 5, a first end of the second line may connect to the first line at a midpoint of the first line and a second end of the second line may be a distance away from the first line, the distance being equal to a puncture height above the anatomical landmark (e.g., the mitral valve annulus). Generating the annotation overlay may further comprise generating a third line perpendicular to the second line, as noted at 916. Also as described with respect to FIG. 5, the third line may extend towards the direction of the first landmark point (e.g., towards the first end of the mitral valve annulus). In examples in which the annotation overlay indicates a position of a TSP, the third line may extend at least to the interatrial septum to indicate the position of the TSP.

At 918, method 900 includes determining the height of the third line above the first line. The height of the third line above the first line may be the length of the second line given that the second line is disposed between and perpendicular to the first and third lines. The height may be determined in various ways. For example, a preset height may be stored in memory and may be obtained, as noted at 920. The preset height may be the initial height to which the annotation overlay is set when initially displayed within the GUI. Alternatively, or additionally, a third user input may be received indicating the height, as noted at 922. As is described with respect to FIGS. 5 and 6, the third user input may be a user input to the third line changing the position of the third line, which inherently alters the length of the second line. The third user input may also be an inputted height value to a height element within the GUI. The displayed annotation overlay may be updated in real-time for both types of inputs, wherein the third line may be dragged to different heights in the first type of user input and/or may update to an inputted height value in response to the second type of user input. The third line at a designated height may indicate a third landmark point. In some examples, the third landmark point may be a TSP position along the interatrial septum.

The annotation overlay may be displayed within the GUI on top of the displayed image respective to the various landmarks. The annotation overlay, during the initial display and while it may be edited via user inputs, may be displayed as a first color.

At 924, method 900 includes saving the annotation data to memory. The annotation data may include respective landmark point positions and line lengths, including the height of the third line above the first line (e.g., the length of the second line). The annotation data may be saved for the corresponding displayed image and may be accessed at a later time by the user or other users of the system in order to view the annotation overlay on the image within the GUI. In some examples, the annotation data may be saved independent of the corresponding displayed image to which it was initially generated and may be displayed later on other images. For example, the annotation overlay may be generated and displayed on top of a pre-operative echocardiogram image. The data of the annotation overlay may then be saved to memory and may be accessed and displayed on an intra-operative echocardiogram image. In this way, a pre-operative TSP position/height may be determined based on pre-operative imaging and the TSP position/height may be compared to a real-time live intra-operative echocardiogram image showing tenting of the septum from a puncturing device to ensure that the position of the tenting aligns with the TSP position/height. As such, improper positioning of the puncturing device may be reduced.

Overall, the methods for annotating images as herein described may reduce time spent by physicians in measuring heights between anatomical landmarks. In particular for the use case of TSP herein presented, the method includes generating perpendicular lines, which may reduce improper positioning and/or measurement of a TSP height. Further, because the annotation is interactive with user inputs, a reduced amount of user inputs may be used to determine the TSP height, therein reducing time spent by the user.

Turning now to FIG. 10, a flowchart illustrating a method 1000 for automated generation of an annotation overlay is shown. The method 1000 will be described with relation to the systems depicted in FIGS. 1-2, but it should be understood that similar methods may be used with other systems without departing from the scope of this disclosure. The method 1000 may be carried out via instructions stored in non-transitory memory of one or more computing devices. For example, method 1000 may be carried out by the processor 204 based on instructions stored in non-transitory memory 206 of the image processing system 200 of FIG. 2.

At 1002, method 1000 includes obtaining image data of a patient. The image data may be 2D or 3D image data acquired by an imaging system. For example, the image data may be acquired by the ultrasound imaging system 100 of FIG. 1 and may comprise a plurality of 2D ultrasound images. In some examples, the plurality of 2D ultrasound images may be acquired over a period of time, for example images of a heart over a period of several heartbeats. In some examples, the image data may be obtained from a database that stores the data in memory, such as a PACS system. In other examples, the image data may be obtained by the imaging system in real-time.

At 1004, method 1000 includes displaying image(s) of the image data within a GUI, such as the first GUI 300 and/or the second GUI 400 described above. The GUI may be displayed by a display device in communication with the one or more computing devices, such as the display device 118 of FIG. 1 and/or the display device 234 of FIG. 2. The image(s) may be displayed within a viewport of the GUI, as is described above.

At 1006, method 1000 includes determining a first anatomical landmark point. Similar to as described with respect to FIG. 9, the first anatomical landmark point may be a first mitral valve annulus point, though other anatomical landmark points are possible. As is described above, machine learning algorithms, e.g., a segmentation based network, may be trained for the first anatomical landmark point and based on the machine learning algorithm and the obtained image data, the first anatomical landmark point may be detected, as noted at 1008.

At 1010, method 1000 includes determining a second anatomical landmark point. The second anatomical landmark point may be a second mitral valve annulus point, though other anatomical landmark points are possible. Similar to as described above, the second anatomical landmark point may be detected based on a machine learning algorithm trained for the second anatomical landmark point, as noted at 1012.

At 1014, method 1000 includes generating an annotation overlay. As is described in detail with respect to FIGS. 5 and 9, the annotation overlay may comprise a first line generated between the first and second anatomical landmark points, a third line parallel to the first line, and a second line perpendicular to both the first and third lines and positioned between the first and third lines. The length of the second line may define a height of the third line above the first line, in some instances this height may be a puncture height for a TSP. In order to generate the annotation, overlay as such, in some examples, a preset height of the third line above the first line (e.g., the length of the second line) may be obtained, as noted at 1012. The preset height may be stored in memory and used as a default for generating the annotation overlay. Additionally, or alternatively, the height may be determined via user inputs, as noted at 1014. As described above, the user inputs may be an input changing the position of the third line to alter the length of the second line and/or an input to a height element indicating a height in cm of the third line above the first line.

In other examples, the position of a tenting in the interatrial septum may be detected in order to determine the height, as noted at 1016. In such examples, images acquired intra-operatively may be acquired as a device is maneuvered through vessels into the heart. Once in the heart, the device may be positioned against the septum so as to cause the septum to bulge or tent. The position of the tenting may be a presumed position of the TSP. The tenting may be detected, in some examples, based on a machine learning algorithm. The position of the tenting as such determined may then define the position of the third line and as a result may define the height of the third line above the first line. The third line may always be parallel to the first line and as such the second line defining the height of the third line may always be perpendicular to the first line (e.g., approximating the mitral valve annulus) and the third line (e.g., defining a position of the TSP). The position of the tenting may act as a third anatomical landmark for the annotation overlay.

At 1018, method 1000 includes displaying the annotation overlay on the image in the GUI. In some examples, the annotation overlay may be displayed in response to determination/detection of each of the anatomical landmarks, including the first anatomical landmark (e.g., the first mitral valve annulus point), the second anatomical landmark (e.g., the second mitral valve annulus point), and the third anatomical landmark (e.g., the tenting and/or TSP position). In this way, the annotation overlay may be displayed over respective anatomy showing the positions of each of the landmarks therein. Similar to as described with respect to FIGS. 5, 6, and 9, the annotation overlay may be editable via user inputs. Further, data of the annotation may be saved to memory to be accessed later, by different users, and/or for different images, as is described with respect to FIG. 9.

A technical effect of the systems and methods provided herein is that time spent in measuring heights/distances between anatomical landmarks may be reduced. As the methods include generating two parallel lines with a third line between that is perpendicular to the two parallel lines, the third line may allow for accurate measurement of height of a second anatomical landmark above a first anatomical landmark. In this use case example described herein, the methods and systems may allow for more accurate positioning of a puncture device during a procedure. In this way, time spent in determining the puncture height may be reduced and need for repositioning of annotation lines may be reduced. Further, the generated annotation overlays are interactive via user inputs. Therefore, processing power may be reduced by reducing the number of inputs needed to generate an accurate annotation overlay. The reduction of number of inputs may further reduce time spent by the user in generating the annotation overlay when measuring height.

The disclosure also provides support for a method, comprising: obtaining an image dataset of a patient acquired by an imaging system, displaying an image of the image dataset within a graphical user interface (GUI) on a display device communicatively coupled to the imaging system, determining a first anatomical landmark point of the image, determining a second anatomical landmark point of the image, generating an annotation overlay based at least in part on the first and second anatomical landmark points, displaying the annotation overlay over the image in the GUI, and saving data of the annotation overlay to memory. In a first example of the method, the annotation overlay comprises a first line connecting the first and second anatomical landmark points, a second line perpendicular to the first line, and a third line perpendicular to the second line, wherein the second line connects the first line to the third line and defines a height of the third line above the first line. In a second example of the method, optionally including the first example, determining the first anatomical landmark point comprises receiving a first user input to the GUI and determining the second anatomical landmark point comprises receiving a second user input to the GUI. In a third example of the method, optionally including one or both of the first and second examples, the first anatomical landmark point is a first mitral valve annulus point indicating a first end of a mitral valve annulus and the second anatomical landmark point is a second mitral valve annulus point indicating a second end of the mitral valve annulus. In a fourth example of the method, optionally including one or more or each of the first through third examples, the first line connecting the first anatomical landmark point to the second anatomical landmark point approximates a position of the mitral valve annulus. In a fifth example of the method, optionally including one or more or each of the first through fourth examples, the third line defines a third anatomical landmark point. In a sixth example of the method, optionally including one or more or each of the first through fifth examples, the third anatomical landmark point is a position of a transseptal puncture. In a seventh example of the method, optionally including one or more or each of the first through sixth examples, the height of the third line above the first line is defined by a length of the second line. In a eighth example of the method, optionally including one or more or each of the first through seventh examples, the method further comprises: determining the height of the third line above the first line based on one of one or more user inputs, a preset height, and a detected tenting position. In a ninth example of the method, optionally including one or more or each of the first through eighth examples, the first and second anatomical landmarks are determined in an automated manner based on machine learning algorithms.

The disclosure also provides support for a system, comprising: a computing device communicatively coupled to an imaging system configured to generate image data of a patient and to a display device, the computing device configured with instructions in non-transitory memory that when executed cause the computing device to: display an image acquired by the imaging system in a graphical user interface (GUI) on the display device, generate an annotation in response to determination of a plurality of landmark points, display the annotation as an overlay on the image, and save the annotation to memory, wherein the annotation comprises a first line between a first landmark point of the plurality of landmark points and a second landmark point of the plurality of landmark points, a second line perpendicular to the first line, and a third line perpendicular to the second line and defining a third landmark point, wherein the third landmark point is a height above the first line defined by a length of the second line. In a first example of the system, the length of the second line is determined based on one or more user inputs. In a second example of the system, optionally including the first example, the imaging system is an ultrasound imaging system configured to acquire echocardiogram images. In a third example of the system, optionally including one or both of the first and second examples, the saved annotation is accessible to be overlaid on one or more images of the patient. In a fourth example of the system, optionally including one or more or each of the first through third examples, the first landmark point is a first mitral valve annulus point, the second landmark point is a second mitral valve annulus point, and the third landmark point is a transseptal puncture position.

The disclosure also provides support for a method, comprising: obtaining one or more images acquired by an imaging system, displaying a first image of the one or more images in a graphical user interface (GUI) on a display device, determining a first mitral valve annulus point of the first image, determining a second mitral valve annulus point of the first image, generating a transseptal puncture (TSP) annotation based on the first and second mitral valve annulus point and a TSP height, wherein the TSP annotation comprises a first line perpendicular to a second line and parallel to a third line, the first line extending between the first and second mitral valve annulus points and the second line extending between the first line and the third line, wherein the third line extends over an interatrial septum, and displaying the TSP annotation as an overlay on the first image. In a first example of the method, the first and second mitral valve annulus point are determined in an automated manner. In a second example of the method, optionally including the first example, the one or more images acquired by the imaging system are acquired and displayed in real-time. In a third example of the method, optionally including one or both of the first and second examples, the TSP height is defined by a detected position of a tenting in the interatrial septum. In a fourth example of the method, optionally including one or more or each of the first through third examples, the third line has a first length at least as long as a second length of the first line.

When introducing elements of various embodiments of the present disclosure, the articles “a,” “an,” and “the” are intended to mean that there are one or more of the elements. The terms “first,” “second,” and the like do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangement. Thus, while the information has been described above with particularity and detail in connection with what is presently deemed to be the most practical and preferred aspects, it will be apparent to those of ordinary skill in the art that numerous modifications, including, but no limited to, form, function, manner of operation and use may be made without departing from the principles and concepts set forth herein. Also, as used herein, the examples and embodiments, in all respects, are meant to be illustrative only and should not be construed to be limiting in any manner.

1. A method, comprising:
obtaining an image dataset of a patient acquired by an imaging system;
displaying an image of the image dataset within a graphical user interface (GUI) on a display device communicatively coupled to the imaging system;
determining a first anatomical landmark point of the image;
determining a second anatomical landmark point of the image;
generating an annotation overlay based at least in part on the first and second anatomical landmark points;
displaying the annotation overlay over the image in the GUI; and
saving data of the annotation overlay to memory.

2. The method of claim 1, wherein the annotation overlay comprises a first line connecting the first and second anatomical landmark points, a second line perpendicular to the first line, and a third line perpendicular to the second line, wherein the second line connects the first line to the third line and defines a height of the third line above the first line.

3. The method of claim 1, wherein determining the first anatomical landmark point comprises receiving a first user input to the GUI and determining the second anatomical landmark point comprises receiving a second user input to the GUI.

4. The method of claim 1, wherein the first anatomical landmark point is a first mitral valve annulus point indicating a first end of a mitral valve annulus and the second anatomical landmark point is a second mitral valve annulus point indicating a second end of the mitral valve annulus.

5. The method of claim 4, wherein the first line connecting the first anatomical landmark point to the second anatomical landmark point approximates a position of the mitral valve annulus.

6. The method of claim 1, wherein the third line defines a third anatomical landmark point.

7. The method of claim 6, wherein the third anatomical landmark point is a position of a transseptal puncture.

8. The method of claim 1, wherein the height of the third line above the first line is defined by a length of the second line.

9. The method of claim 1, further comprising determining the height of the third line above the first line based on one of one or more user inputs, a preset height, and a detected tenting position.

10. The method of claim 1, wherein the first and second anatomical landmarks are determined in an automated manner based on machine learning algorithms.

11. A system, comprising:
a computing device communicatively coupled to an imaging system configured to generate image data of a patient and to a display device, the computing device configured with instructions in non-transitory memory that when executed cause the computing device to:
display an image acquired by the imaging system in a graphical user interface (GUI) on the display device;
generate an annotation in response to determination of a plurality of landmark points;
display the annotation as an overlay on the image; and
save the annotation to memory, wherein the annotation comprises a first line between a first landmark point of the plurality of landmark points and a second landmark point of the plurality of landmark points, a second line perpendicular to the first line, and a third line perpendicular to the second line and defining a third landmark point, wherein the third landmark point is a height above the first line defined by a length of the second line.

12. The system of claim 11, wherein the length of the second line is determined based on one or more user inputs.

13. The system of claim 11, wherein the imaging system is an ultrasound imaging system configured to acquire echocardiogram images.

14. The system of claim 11, wherein the saved annotation is accessible to be overlaid on one or more images of the patient.

15. The system of claim 11, wherein the first landmark point is a first mitral valve annulus point, the second landmark point is a second mitral valve annulus point, and the third landmark point is a transseptal puncture position.

16. A method, comprising:
obtaining one or more images acquired by an imaging system;
displaying a first image of the one or more images in a graphical user interface (GUI) on a display device;
determining a first mitral valve annulus point of the first image;
determining a second mitral valve annulus point of the first image;
generating a transseptal puncture (TSP) annotation based on the first and second mitral valve annulus point and a TSP height, wherein the TSP annotation comprises a first line perpendicular to a second line and parallel to a third line, the first line extending between the first and second mitral valve annulus points and the second line extending between the first line and the third line, wherein the third line extends over an interatrial septum; and
displaying the TSP annotation as an overlay on the first image.

17. The method of claim 16, wherein the first and second mitral valve annulus point are determined in an automated manner.

18. The method of claim 17, wherein the one or more images acquired by the imaging system are acquired and displayed in real-time.

19. The method of claim 16, wherein the TSP height is defined by a detected position of a tenting in the interatrial septum.

20. The method of claim 16, wherein the third line has a first length at least as long as a second length of the first line.
